(19) United States
(12) Patent Application Publication
(10) Pub. No.: US 2022/0406437 A1
(43) Pub. Date: Dec. 22, 2022

(54) METHODS AND SYSTEMS FOR DETERMINING AND PROVIDING RENAL THERAPY

(71) Applicants: Fresenius Medical Care Holdings, Inc., Waltham, MA (US); Fresenius Medical Care Deutschland GMBH, Bad Homburg (DE)

(72) Inventors: Caitlin Monaghan, Arlington, MA (US); Len Usvyat, Boston, MA (US); Felix Adam, Waltham, MA (US); Dinesh Chatoth, Waltham, MA (US); Michael Kraus, Waltham, MA (US)

(21) Appl. No.: 17/843,972

(22) Filed: Jun. 18, 2022

Related U.S. Application Data
(60) Provisional application No. 63/212,314, filed on Jun. 18, 2021.

(51) Int. Cl. G16H 20/40; G16H 40/60; G16H 50/30; A61M 1/14

(52) U.S. Cl. CPC G16H 20/40; G16H 40/60; G16H 50/30; A61M 1/14

(57) ABSTRACT

The described technology may include processes to model a modality status in patients and/or patient populations. In one embodiment, a method may include a modality analysis. The method may include, via a processor of a computing device: determining a modality analysis model configured to determine a modality status of a patient, the modality status configured to indicate a probability of a transition from a first modality to a second modality, and generating the modality status via the modality analysis model using patient information associated with the patient. Other embodiments are described.

### CROSS REFERENCE TO RELATED APPLICATIONS

The present application claims the benefit of and priority to U.S. Provisional Patent Application Ser. No. 63/212,314, filed Jun. 18, 2021, the disclosure of which is incorporated herein by reference.

### BACKGROUND

Renal replacement therapy may be performed using various modalities, including, without limitation, peritoneal dialysis (PD) or hemodialysis (HD) (for instance, home hemodialysis (HHD) or in-center hemodialysis (ICHD)). Selection of the optimal dialysis modality is central to positive patient experiences and outcomes on renal replacement therapy. There are multiple advantages to initiating RRT with PD, including preservation of residual renal function and vascular accesses, and overall quality of life due to preservation of lifestyle, independence, schedule flexibility, and hospital avoidance. Although PD is an optimal modality for many patients, clinical complications such as catheter malfunctions, peritonitis, and ultrafiltration failure do occur, leading some patients to transition from PD to another modality (for instance, ICHD). Typically, a patient and healthcare providers may not be aware that a transition is required or imminent until a serious health concern (for instance, a hospitalization, a cardiac event, an infection, complications from fluid overload due to inadequate dialysis, and/or the like) has occurred. Accordingly, an urgent transition from PD to ICHD is costly and incurs a high hospitalization rate.

It is with respect to these and other considerations that the present improvements may be useful.

### BRIEF DESCRIPTION OF THE DRAWINGS

FIG. 1 illustrates a first exemplary operating environment in accordance with the present disclosure;

FIG. 2 illustrates patient information in accordance with the present disclosure;

FIG. 3 illustrates a first modality status information report in accordance with the present disclosure;

FIG. 4 illustrates a second modality status information report in accordance with the present disclosure;

FIG. 5 illustrates a computational model in accordance with the present disclosure;

FIG. 6 illustrates example computational model performance in accordance with the present disclosure;

FIG. 7 illustrates another example computational model performance in accordance with the present disclosure;

FIG. 8A, 8B, 8C, 8D, and 8E illustrate an example care map in accordance with the present disclosure; and

FIG. 9 illustrates an embodiment of a computing architecture in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

The described technology is generally directed to processes, systems, and methods for dialysis modality analysis processes. In accordance with various aspects of the described embodiments, a method of modality analysis may include determining a modality status for a patient based on one or more patient characteristics. In some embodiments, a modality status may provide an indicator of a likelihood that a patient may require a transition from a first modality to a second modality. For instance, a modality status of a patient may indicate whether a patient may require a transition from PD to HD (for instance, HHD and/or ICHD). In various embodiments, modality analysis processes may include providing a physiology-based computational model configured to model the modality status of a patient. For instance, a modality analysis process may, using a computational model according to some embodiments, determine patients who are at risk for dropping out of PD over one or more analysis durations, such as a short-term duration (1-3 months) and long-term duration (3-6 months).

Conventional techniques have proven inadequate for the diagnosis and treatment of patients at risk of leaving a dialysis modality. Typically, a transition occurs without adequate notice to the patient and/or healthcare provider to adequately mitigate the negative effects of the transition. If a patient and/or healthcare provider had a more advanced and detailed warning of potential health issues that may lead to a transition, the transition may be avoided or the transition may be managed better with a better outcome for the patient.

Accordingly, some embodiments may provide computational models operative to perform a modality analysis process. In various embodiments, the modality analysis process may operate to predict or otherwise determine a transition. For example, a modality analysis process may determine a potential transition from PD to HHD or ICHD. In various embodiments, a modality analysis process may model various transition risks, such as transition risks over different analysis durations, including, without limitation, an immediate transition risk in the following 1-3 calendar months or a risk of patient transitioning out of PD in the next 3 to 6 months. In general, a transition may include being on a new modality for a minimum duration, such as 30 days.

In some embodiments, the computational models may operate using advanced analytics to provide a tool to better identify patients at risk of leaving a modality. The modality analysis process may use the computational models to determine a modality status to, inter alfa, avoid controllable PD discharges by identifying risks earlier, facilitate transitions to HD (ICHD or HHD) through earlier and more accurate identification of technique failure, and/or allocate resources towards patients more likely to drop to ensure proper education and access planning.

In some embodiments, the computational models may be or may include one or more types of models, including, without limitation, artificial intelligence (AI) models, machine learning (ML) models, deep learning (DL) models, neural network (NN) models, tree-based models (for instance, decision trees), gradient boosting frameworks (for instance, XGBoost) combinations thereof, variations thereof, and/or the like, now known or developed in the future. Computational models may be trained using various training data. For example, a model may be trained using relatively recent (for instance, 2016-2019) data of about 53,000 patients over 800,000 patient months, with a number of predictors (variables or patient information) of about 230 (with about 40 “core” predictors). Embodiments are not limited in this context. In some embodiments, a computational model may be configured as an XGBoost ML model. In various embodiments, a computational model may be trained to identify any modality change (e.g., from PD to HD) lasting more than 30 days. In some embodiments, a computational model may be configured to identify, among other things, different drivers behind urgent drops (1-3 months) and long-term predictions (3-6 months).

Although PD, HD, HHD, and/or ICHD, and transitioning from PD to a form of HD, are used in examples, embodiments are not so limited. As the modality analysis processes may be used to analyze a transition among these modalities and/or different modalities using computational models according to some embodiments.

Some embodiments may provide, inter alfa, clinical decision support tool(s) designed to optimize patient care through management of dialysis modalities, such as but not limited to peritoneal dialysis (PD), home hemodialysis (HHD), and in-center hemodialysis (ICHD). Modality analysis processes may use a modality management tool that incorporates operational, financial, and clinical data to optimize a patient's time on their current modality and/or provide insight into likelihood of leaving the modality to enable smoother transitions through long term planning of care. Using machine learning and advanced analytical techniques, this tool integrates vast amounts of data from numerous sources including but not limited to laboratory values, hospitalizations, free text clinical notes, medical history, and comprehensive assessments. These data are integrated over time to assess trends, resulting in two risk scores for short (1-3 month) and long-term (3-6 month) discharge from the PD modality. The management tool may be integrated into the workflow of care providers to enable advanced and fully informed support for patient modality management and patient care in general.

Modality analysis processes, computational models and treatment methods of using modality analysis processes and computational models may provide multiple technological advantages over conventional techniques, including advances in computing technology. In one non-limiting example of a technological advantage, modality analysis processes may operate to accurately and efficiently model modality transition indications in a manner not available using existing techniques. In another non-limiting technological advantage, modality analysis processes may provide a causality link to identify patient-specific causes of a modality transition in patients and/or patient populations. Accordingly, using modality analysis processes according to some embodiments, a healthcare team may focus on the causal link to potentially avoid the transition. Therefore, in another non-limiting technological advantage, modality analysis processes may provide information and advanced warning to a healthcare team to provide interventional treatment to a patient to avoid a transition or facilitate a transition to a new modality with better patient care and outcomes than available using existing technologies.

Processes, techniques, methods, systems, and/or the like described in the present disclosure may be integrated into various practical applications. For example, modality analysis processes and computational models according to some embodiments may determine one or more causal factors for requiring a transition or potential of a patient and/or patient population from a first modality to a second modality. In an additional example, modality analysis processes and computational models may provide an efficient and accurate process for determining the current clinical pathways for modality management and identify opportunities for using data and analytics to identify at-risk patients and target possible interventions.

Additional technological advantages and integrations of embodiments into practical applications are described in, and would be known to those of skill in the art in view of, the present disclosure.

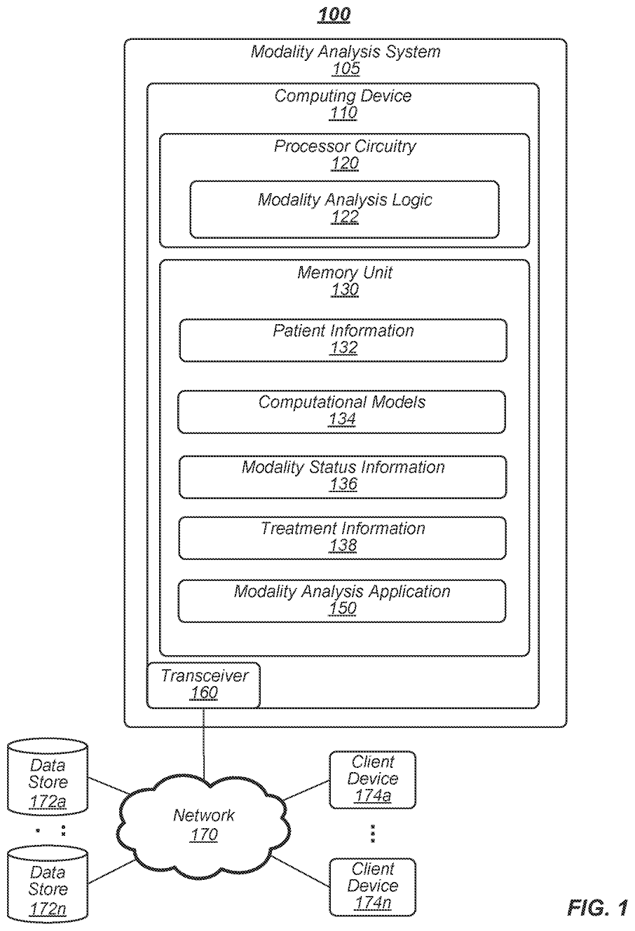

FIG. 1 illustrates an example of an operating environment 100 that may be representative of some embodiments. As shown in FIG. 1, operating environment 100 may include a modality analysis system 105. In various embodiments, modality analysis system 105 may include a computing device 110 communicatively coupled to network 170 via a transceiver 160. In some embodiments, computing device 110 may be a server computer or other type of computing device.

Computing device 110 may be configured to manage, among other things, operational aspects of a modality analysis process according to some embodiments. Although only one computing device 110 is depicted in FIG. 1, embodiments are not so limited. In various embodiments, the functions, operations, configurations, data storage functions, applications, logic, and/or the like described with respect to computing device 110 may be performed by and/or stored in one or more other computing devices (not shown), for example, coupled to computing device 110 via network 170 (for instance, one or more of client devices 174a-n). A single computing device 110 is depicted for illustrative purposes only to simplify the figure. Embodiments are not limited in this context.

Computing device 110 may include a processor circuitry that may include and/or may access various logics for performing processes according to some embodiments. For instance, processor circuitry 120 may include and/or may access a modality analysis logic 122. In some embodiments, modality analysis logic 122 may operate to perform modality analysis processes according to some embodiments.

Processing circuitry 120, modality analysis logic 122, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 900. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a NN model or application, a tree-based model or application, a proportional-integral-derivative (PID) controller, variations thereof, combinations of any of the foregoing, and/or the like.

Although modality analysis logic 122 is depicted in FIG. 1 as being within processor circuitry 120, embodiments are not so limited. For example, modality analysis logic 122 and/or any component thereof may be located within an accelerator, a processor core, an interface, an individual processor die, implemented entirely as a software application (for instance, a modality analysis application 150) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 130 may store various types of information and/or applications for a modality analysis process according to some embodiments. For example, memory unit 130 may store patient information 132, computational models 134, modality information 136, treatment information 138, and/or a modality analysis application 150. In some embodiments, some or all of patient information 132, computational models 134, modality information 136, treatment information 138, and/or a modality analysis application 150 may be stored in one or more data stores 172a-n accessible to computing device 110 via network 170. For example, one or more of data stores 172a-n may be or may include a health information system (HIS), an electronic medical records (EMR) system, a dialysis information system (DIS), a picture archiving and communication system (PACS), a Centers for Medicare and Medicaid Services (CMS) database, U.S. Renal Data System (USRDS), a proprietary database, and/or the like.

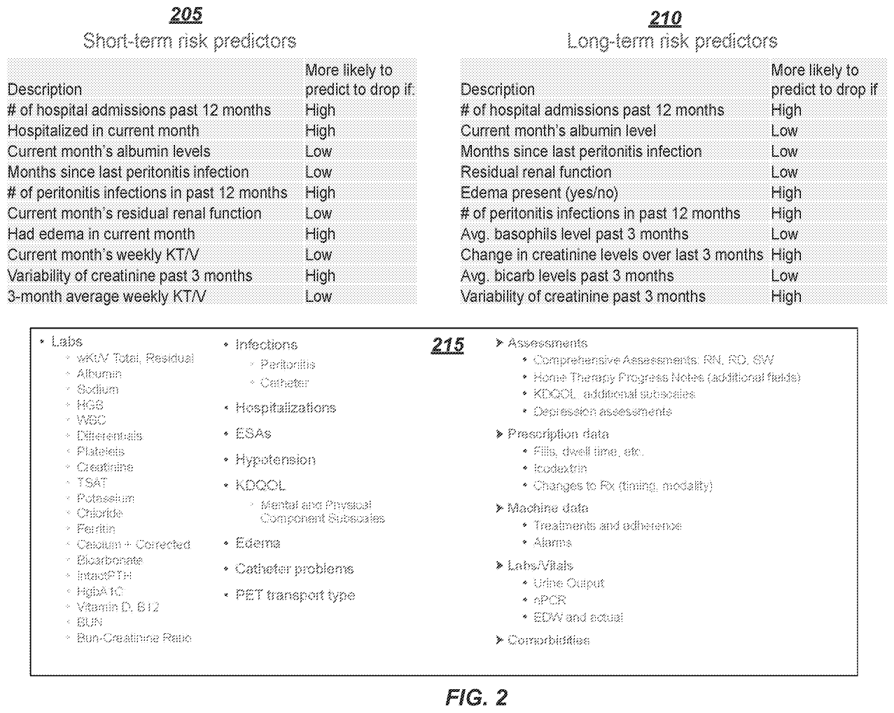

Patient information 132 may include characteristics of a patient and/or a patient population that may be relevant to determining a modality status according to some embodiments. FIG. 2 depicts illustrative and non-limiting examples of patient information 132. For example, patient information 132 may include predictors driving risk for one or more timeframes, such as a short-term time frame or a long-term time frame. As shown in FIG. 2, for instance, patient information 132 may be used for predictions for certain time frames, such as short-term risk predictors 205 and long-term risk predictors 210. In addition, patient information 132 may be associated with a risk predictor, such as a high or low value indicating if a patient is more likely to drop or transition (values may include numerical thresholds or other types of values). Additional patient information 215 may also be used according to some embodiments.

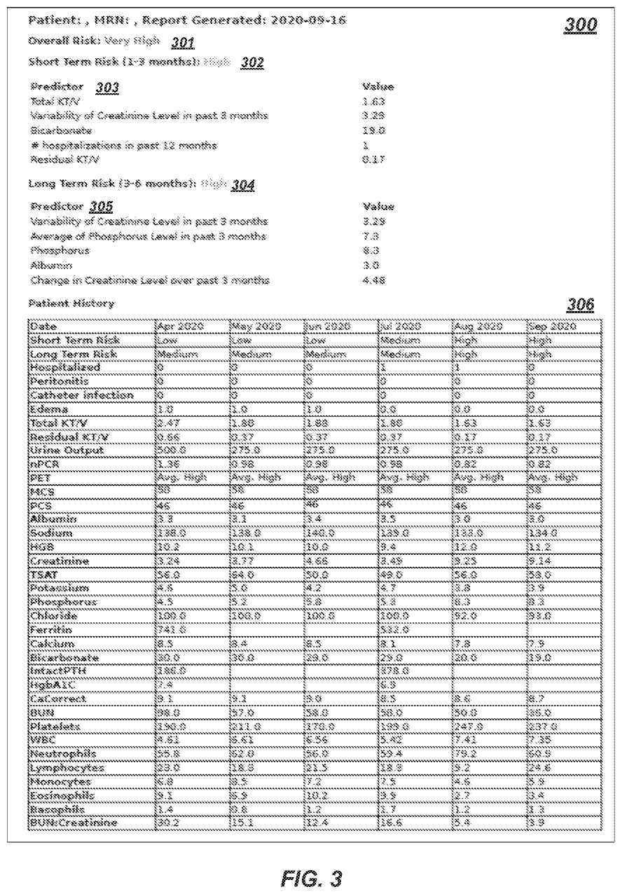

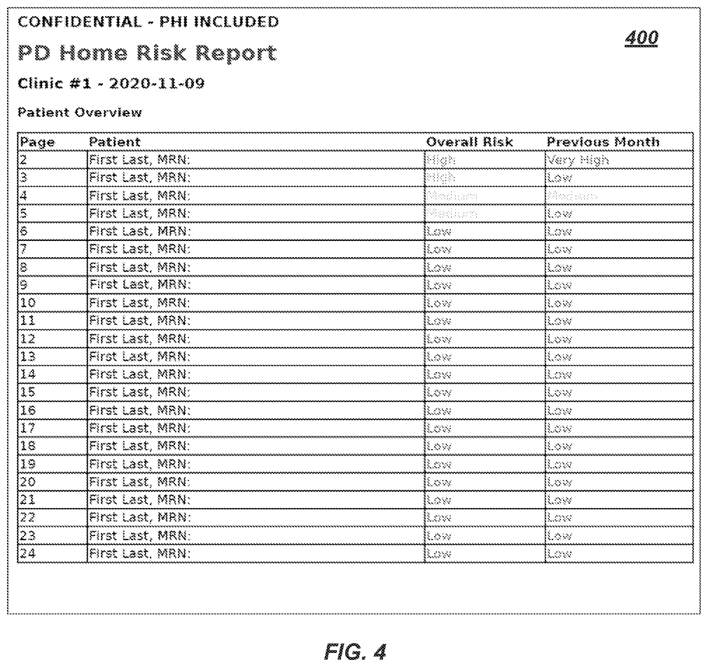

In some embodiments, modality analysis application 150 may use one or more computational models 136 to analyze patient information 132 to determine modality status information 136 and/or treatment information 138. In some embodiments, modality status information 136 may be or may include a report, a score, a treatment recommendation, and/or the like. FIG. 3 depicts an illustrative and non-limiting example of a report 300 of modality status information. As shown in FIG. 3, report 300 may be individualized for one patient and may include patient history 306 (which depicts additional patient information 132). In some embodiments, report 300 may include various risk predictions, such as an overall risk 301, a short-term risk 302, and a long-term risk 304. Risks may be associated with predictors 303, 305 that provide one or more specific items of patient information 132 that lead to the prediction. Accordingly, a healthcare provider may recommend and/or administer treatment that addresses a predictor to stave off a transition and/or to facilitate a better transition outcome. FIG. 4 depicts an illustrative and non-limiting example of a report 400 of modality status information. As shown in FIG. 4, report 400 may be configured for a group of patients, for example, receiving care through a common healthcare provider or professional. In this manner, a healthcare provider may view a status of a group of patients, for example, to prioritize healthcare interventions based on risk. In some embodiments, report 400 may include trends (for instance, two or more months of modality status risk assessments) to determine patient trends. In some embodiments, color, highlighting, or other indicators may be used to communicate risk information. For example, yellow may be used for a Phase 1A (medium overall risk), orange may be used for a Phase 1B (in Phase 1, but consider Phase 2; high overall risk), and red for Phase 2 (very high overall risk).

In some embodiments, computational models may generate a model status, for example, in the form of a risk predictor. In some embodiments, a model status may be or may include a numerical value or score. In other embodiments, a model status may be or may include an identifier, such as “low risk,” “medium risk, “high risk,” and/or the like. A model status may use various types of indications, including numerical, textual, graphical, and/or the like.

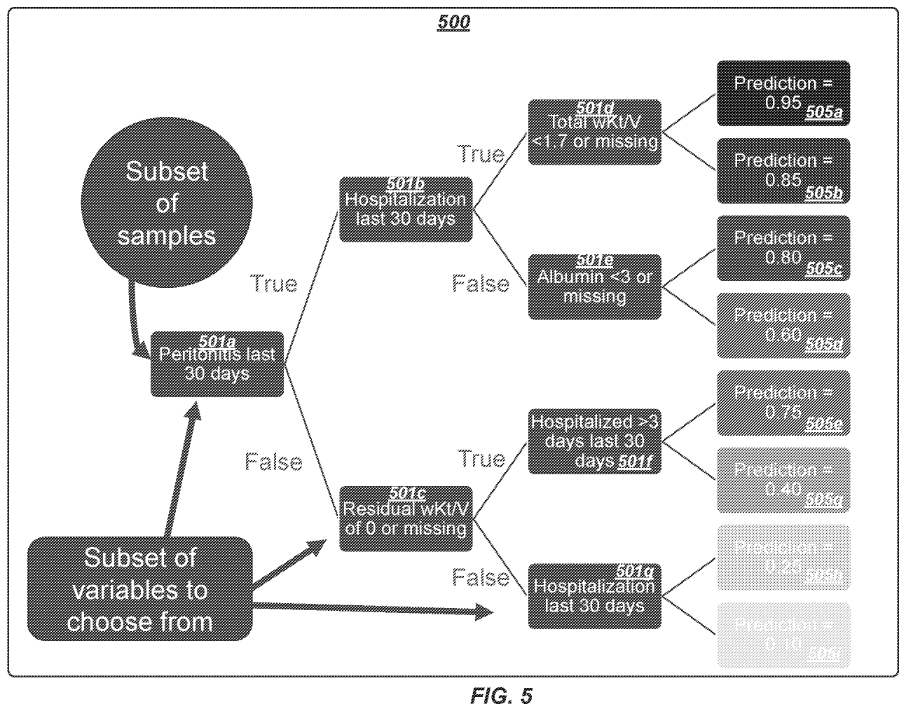

FIG. 5 depicts an illustrative and non-restrictive example of a computational model according to the present disclosure. As shown in FIG. 5, a computational model 500 may be or may include a tree-based model, including, without limitation, a decision tree. In various embodiments, computational model 500 may be formed of nodes 501a-g that may include a subset of samples or variables that may be used to determine a prediction value of nodes 505a-i. In some embodiments, variables of nodes 501a-g may be selected based on their predictive ability for determining a modality status, alone, or in combination with other nodes. For example, node 501f may have predictive value in combination (i.e., as a leaf of node 501c) with another particular node. Implementation of computational model 500 may transition through the variable nodes 501a-f depending on patient information (i.e., for node 501a, whether a patient has had peritonitis in the last 30 days) to arrive at a modality status prediction. For example, if the analysis for nodes 501a, 501b, and 501d are true, a patient may have a prediction of 0.95 of node 505a (or 95% chance) that a transition is likely (for instance, on a scale of 0.0 to 1.0, with 1.0 indicating that a transition is required.

In general, tree-based methods and computational models according to some embodiments provide various advantages including, without limitation, being less prone to overfitting, being able to handle missing values without imputation, being able to handle collinearity, and/or being able to use a larger number of variables compared with conventional methods and/or other computational models.

### EXAMPLE I

### Computational Model Performance

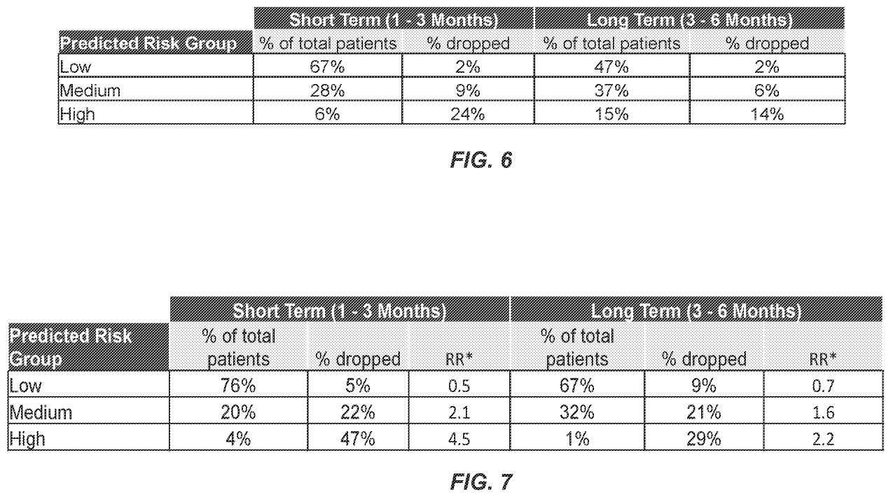

FIGS. 6 and 7 depict illustrative computational model performance using processes according to some embodiments. For example, in FIG. 6, PD patients were grouped into three risk categories: low, medium, and high. In some embodiments, patients may be grouped into categories for different time frame predictions, such as a short-term risk or a long-term risk. In this example, a baseline probability of a transition is about a 5% chance that a randomly chosen patient leaves PD within 1-3 months (3-6 months: 6%). The performance information is for a retrospective analysis of 7000 patients (100,000 patient months). As indicated in FIG. 6, the computational model provides predictive indicators for both short-term and long-term PD transitions (RR is a relative risk calculated based on a baseline probability). In another example, FIG. 7 provides information for a retrospective analysis of 26,277 patients (129,000 patient months) (all PD drops excluding transplants (including deaths, transfers out of FMCNA, etc. as reasons for leaving PD), with a baseline probability of dropping of 10.5 percent chance that a randomly chosen patient leaves PD within 1-3 months (3-6 months: 13%).

### EXAMPLE II

### Care Maps

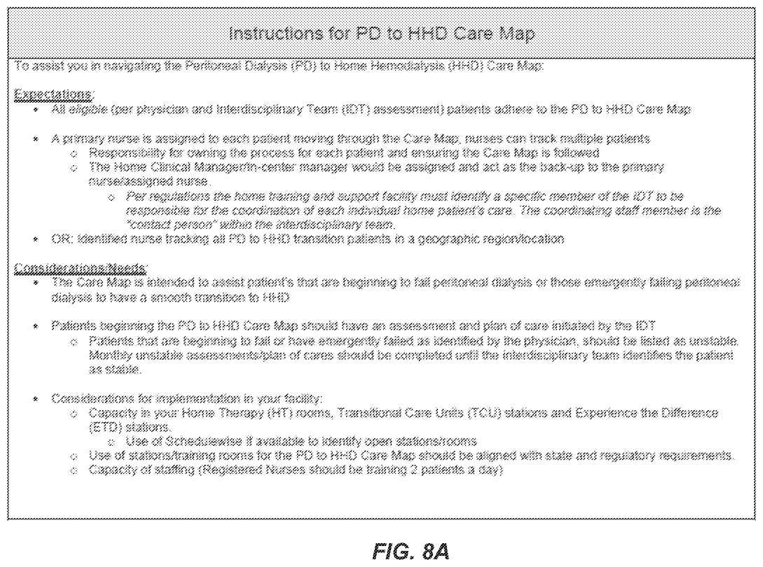

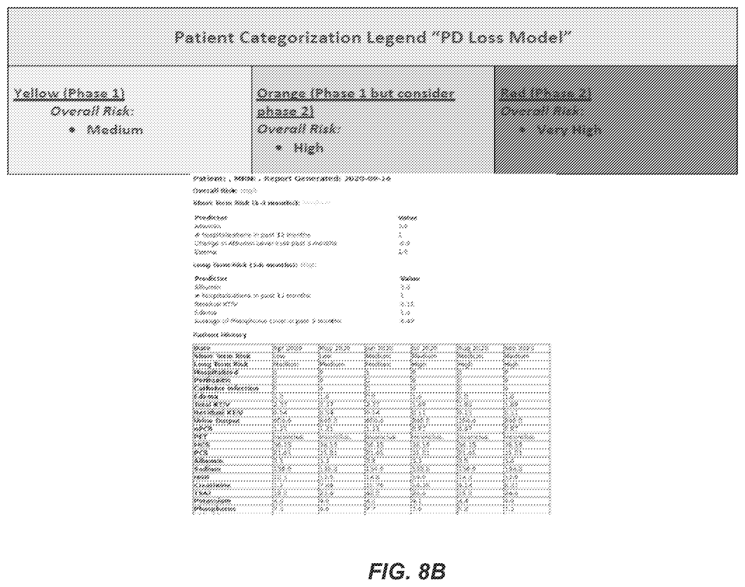

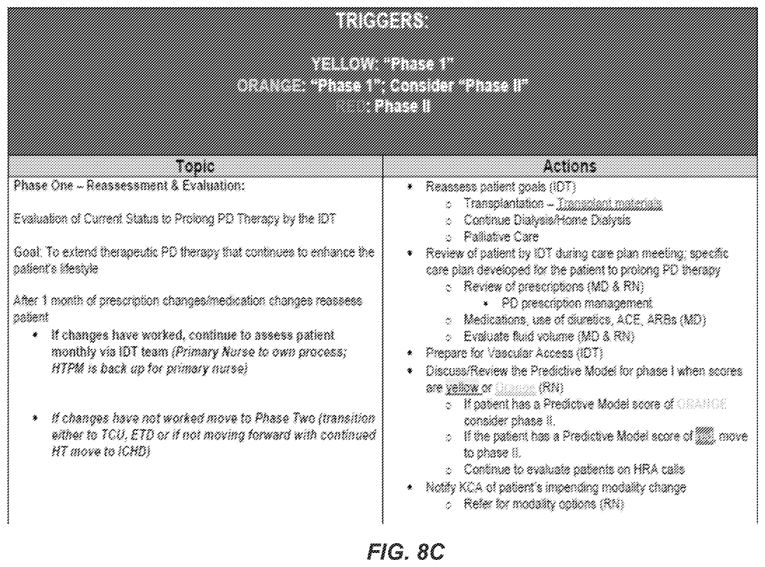

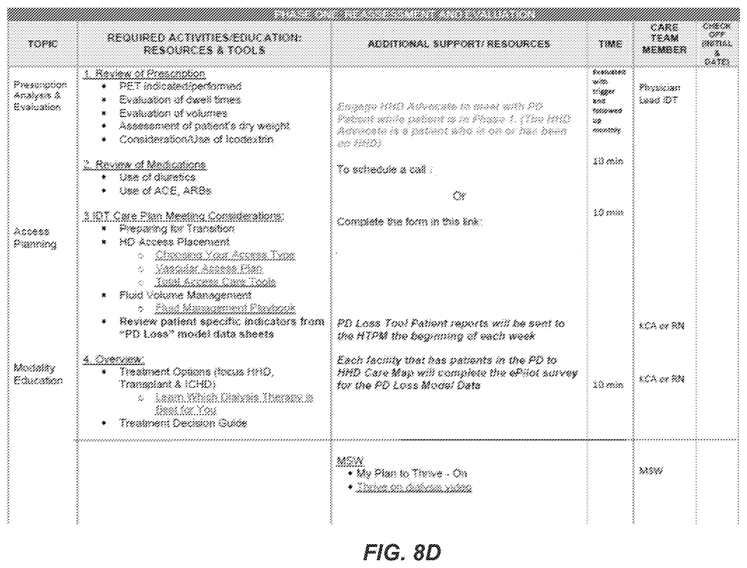

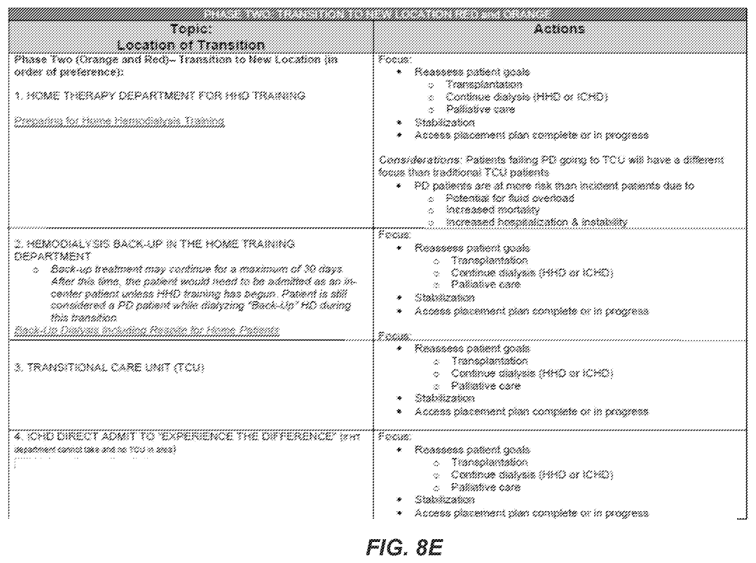

Some embodiments may provide one or more workflows, care plans, or care maps (“Care Map”). In general, a Care Map is intended to assist patients that are beginning to fail peritoneal dialysis or those emergently failing peritoneal dialysis to have a smooth transition to a new modality, such as ICHD or HHD. A Care Map may include or use various phases, such as a Phase One: Reassessment & Evaluation, Phase Two: Transition to a New Location, and/or the like. FIGS. 8A-8E depict an illustrative Care Map according to some embodiments.

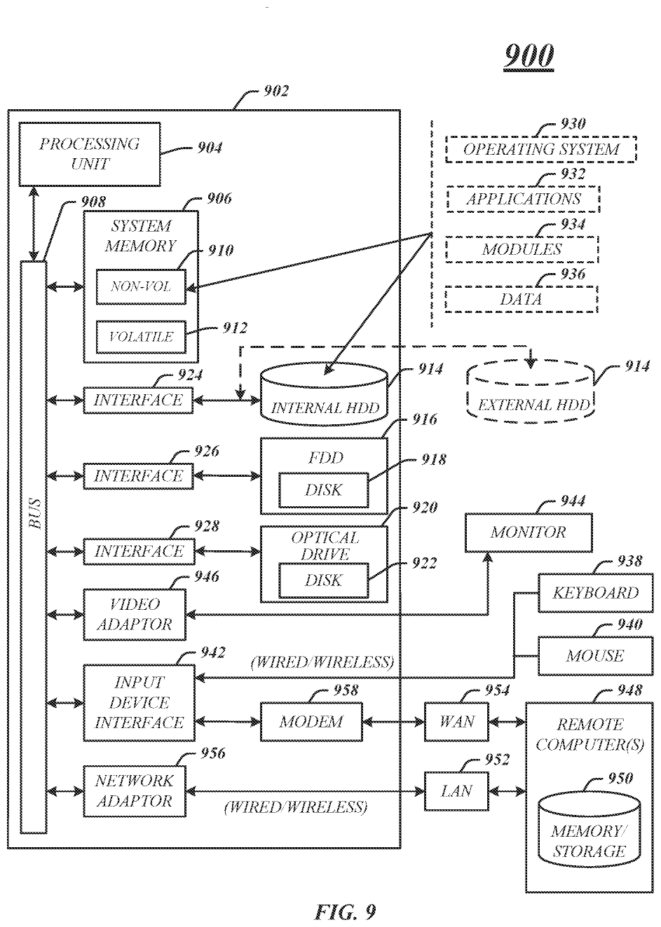

FIG. 9 illustrates an embodiment of an exemplary computing architecture 900 suitable for implementing various embodiments as previously described. In various embodiments, the computing architecture 900 may comprise or be implemented as part of an electronic device. In some embodiments, the computing architecture 900 may be representative, for example, of computing device 110. The embodiments are not limited in this context.

As used in this application, the terms “system” and “component” and “module” are intended to refer to a computer-related entity, either hardware, a combination of hardware and software, software, or software in execution, examples of which are provided by the exemplary computing architecture 900. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 900 includes various common computing elements, such as one or more processors, multi-core processors, co-processors, memory units, chipsets, controllers, peripherals, interfaces, oscillators, timing devices, video cards, audio cards, multimedia input/output (I/0) components, power supplies, and so forth. The embodiments, however, are not limited to implementation by the computing architecture 900.

As shown in FIG. 9, the computing architecture 900 comprises a processing unit 904, a system memory 906 and a system bus 908. The processing unit 904 may be a commercially available processor and may include dual microprocessors, multi-core processors, and other multi-processor architectures.

The system bus 908 provides an interface for system components including, but not limited to, the system memory 906 to the processing unit 904. The system bus 908 can be any of several types of bus structure that may further interconnect to a memory bus (with or without a memory controller), a peripheral bus, and a local bus using any of a variety of commercially available bus architectures. Interface adapters may connect to the system bus 908 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 906 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 9, the system memory 906 can include non-volatile memory 910 and/or volatile memory 912. A basic input/output system (BIOS) can be stored in the non-volatile memory 910.

The computer 902 may include various types of computer-readable storage media in the form of one or more lower speed memory units, including an internal (or external) hard disk drive (HDD) 914, a magnetic floppy disk drive (FDD) 916 to read from or write to a removable magnetic disk 911, and an optical disk drive 920 to read from or write to a removable optical disk 922 (e.g., a CD-ROM or DVD). The HDD 914, FDD 916 and optical disk drive 920 can be connected to the system bus 908 by a HDD interface 924, an FDD interface 926 and an optical drive interface 928, respectively. The HDD interface 924 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 910, 912, including an operating system 930, one or more application programs 932, other program modules 934, and program data 936. In one embodiment, the one or more application programs 932, other program modules 934, and program data 936 can include, for example, the various applications and/or components of computing device 110.

A user can enter commands and information into the computer 902 through one or more wired/wireless input devices, for example, a keyboard 938 and a pointing device, such as a mouse 940. These and other input devices are often connected to the processing unit 904 through an input device interface 942 that is coupled to the system bus 908, but can be connected by other interfaces.

A monitor 944 or other type of display device is also connected to the system bus 908 via an interface, such as a video adaptor 946. The monitor 944 may be internal or external to the computer 902. In addition to the monitor 944, a computer typically includes other peripheral output devices, such as speakers, printers, and so forth.

The computer 902 may operate in a networked environment using logical connections via wired and/or wireless communications to one or more remote computers, such as a remote computer 948. The remote computer 948 can be a workstation, a server computer, a router, a personal computer, portable computer, microprocessor-based entertainment appliance, a peer device or other common network node, and typically includes many or all of the elements described relative to the computer 902, although, for purposes of brevity, only a memory/storage device 950 is illustrated. The logical connections depicted include wired/wireless connectivity to a local area network (LAN) 952 and/or larger networks, for example, a wide area network (WAN) 954. Such LAN and WAN networking environments are commonplace in offices and companies, and facilitate enterprise-wide computer networks, such as intranets, all of which may connect to a global communications network, for example, the Internet.

The computer 902 is operable to communicate with wired and wireless devices or entities using the IEEE 802 family of standards, such as wireless devices operatively disposed in wireless communication (e.g., IEEE 802.19 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. A computer-implemented method of modality analysis, the method comprising, via a processor of a computing device:
determining a modality analysis model configured to determine a modality status of a patient, the modality status configured to indicate a probability of a transition from a first modality to a second modality; and
generating the modality status via the modality analysis model using patient information associated with the patient.

2. The method of claim 1, the first modality comprising peritoneal dialysis (PD) and the second modality comprising one of hemodialysis (HD), home hemodialysis (HHD), or in-center hemodialysis (ICHD).

3. The method of claim 1, the modality status indicating a probability of the transition over a transition duration, the transition duration comprising at least one of 1-3 months or 3-6 months.

4. The method of claim 1, the computational model comprising at least one decision tree of nodes comprising variables of patient information.

5. The method of claim 1, further comprising administering a treatment regimen based on the modality status.

6. (canceled)

7. The method of claim 1, wherein the modality status comprises a report, a score, and/or a treatment recommendation.

8. The method of claim 7, wherein the modality status comprises a report and the report comprises an overall risk, a short-term risk, and a long-term risk.

9. The method of claim 8, wherein the overall risk, the short-term risk, and the long-term risk are generated by the modality analysis model based on predictors indicative of the risk.

10. An apparatus, comprising:
a memory device storing instructions; and
at least one processor coupled to the memory device, the at least one processor configured to execute the instructions to cause the apparatus to:
determine a modality analysis model configured to determine a modality status of a patient, the modality status configured to indicate a probability of a transition from a first modality to a second modality; and
generate the modality status via the modality analysis model using patient information associated with the patient.

11. The apparatus of claim 10, wherein the first modality comprising peritoneal dialysis (PD) and the second modality comprising one of hemodialysis (HD), home hemodialysis (HHD), or in-center hemodialysis (ICHD).

12. The apparatus of claim 10, wherein the modality status indicating a probability of the transition over a transition duration, the transition duration comprising at least one of 1-3 months or 3-6 months.

13. The apparatus of claim 10, the computational model comprising at least one decision tree of nodes comprising variables of patient information.

14. The apparatus of claim 10, the at least one processor further configured to execute the instructions to cause the apparatus to administer a treatment regimen based on the modality status.

15. The apparatus of claim 10, wherein the modality status comprises a report, a score, and/or a treatment recommendation.

16. The apparatus of claim 15, wherein the modality status comprises a report and the report comprises an overall risk, a short-term risk, and a long-term risk.

17. The apparatus of claim 16, wherein the overall risk, the short-term risk, and the long-term risk are generated by the modality analysis model based on predictors indicative of the risk.

18. A non-transitory computer readable medium comprising instructions executable by processing circuitry, which when executed cause an apparatus to:
determine a modality analysis model configured to determine a modality status of a patient, the modality status configured to indicate a probability of a transition from a first modality to a second modality; and
generate the modality status via the modality analysis model using patient information associated with the patient.

19. The non-transitory computer readable medium of claim 18, wherein the first modality comprising peritoneal dialysis (PD) and the second modality comprising one of hemodialysis (HD), home hemodialysis (HHD), or in-center hemodialysis (ICHD).

20. The non-transitory computer readable medium of claim 18, wherein the modality status indicating a probability of the transition over a transition duration, the transition duration comprising at least one of 1-3 months or 3-6 months.

21. The non-transitory computer readable medium of claim 10, further comprising instructions, which when executable by the processing circuitry cause the apparatus to administer a treatment regimen based on the modality status.
